(19) United States
(12) Patent Application Publication
(10) Pub. No.: US 2005/0165433 A1
(43) Pub. Date: Jul. 28, 2005

(54) Trocar having planar fixed septum seal and related methods

(76) Inventors: Gary W. Haberland, Oviedo, FL (US); Brent Van Camp, Orlando, FL (US); Michael Scott Athey, Orlando, FL (US)

Correspondence Address: JEFFREY S. WHITTLE;BRACEWELL & PATTERSON, P.O. BOX 61389, HOUSTON, TX, 77208-1389, US

(21) Appl. No.: 10/879,644

(22) Filed: Jun. 29, 2004

Related U.S. Application Data
(63) Continuation of application No. 10/763,762, filed on Jan. 23, 2004, now abandoned.

(51) Int. Cl. A61B 17/32

(52) U.S. Cl. 606/167

(57) ABSTRACT

A septum valve having peripheries thereof adapted to be fixedly positioned in a valve housing of a trocar system is provided. The septum valve includes a valve body having an opening positioned in a medial portion of the valve body and adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough. The valve body has first and second layers of a fabric material and a layer of elastomeric material positioned between and contacting each of the first and second layers of the fabric material. The septum valve also includes a periphery valve section connected to and extending radially outwardly from peripheries of the valve body and having an outer perimeter thereof adapted to be fixedly connected to the valve housing. The periphery valve section has a plurality of rib members each radially extending substantially an entire distance between an outer perimeter of the valve body and the outer perimeter of the periphery valve section and symmetrically positioned spaced-apart from each other. The periphery valve section has a greater flexibility than the valve body.

### RELATED APPLICATION

This patent application is a continuation of, and claims the benefit of, co-pending U.S. Non-Provisional patent application Ser. No. 10/763,762 filed on Jan. 23, 2004, which hereby is incorporated by reference in its entirety.

### BACKGROUND OF THE INVENTION

1. Field of the Invention

The present invention relates in general to the field of medical devices. More particularly, the present invention relates to trocar systems and methods.

2. Description of Related Art

Trocar systems have been developed over the years for various endoscopic applications in the field of medicine. These trocar systems conventionally include a cannula through which a trocar or obturator or other endoscopic related tool extends. It is known to use one or more valves positioned within or connected to a proximal end of the cannula of a trocar system. Examples of such trocar systems having one or more valves in the cannula thereof can be seen in U.S. Pat. No. 5,226,891 by Bushatz et al. titled “Seal Protection Apparatus,” U.S. Pat. No. 5,308,336 by Hart et al. titled “Seal Protection Mechanism,” U.S. Pat. No. 5,385,553 by Hart et al. titled “Trocar With Floating Septum Seal,” U.S. Pat. No. 5,782,812 by Hart et al. titled “Seal Assembly For Access Device” U.S. Pat. No. 5,443,452 by Hart et al. titled “Seal Assembly For Access Device,” and U.S. Pat. No. 5,209,737 by Ritchart et al. titled “Lever Actuated Septum Seal.” These devices, however, can be bulky and awkward to use and have complex multi-component mechanical valves which can be difficult and expensive to manufacture and can have an increased risk of mechanical failure. The mechanical valves also have little or no flexibility.

Other trocar systems have been developed which are easier to use and have less complex mechanical valves. One example of such trocar system can be seen in U.S. Pat. No. 6,569,119 by Haberland et al. titled “Trocar System Having Cannula with Finger Grips.” These devices provide enhanced gripping and easier handling of the systems. Nevertheless, there is also still a need for alternative cannula and valve configurations for trocar systems, a need for relatively less expensive trocar systems, a need for trocar systems with better performance, a need for more flexible trocar systems and valves which enhance handling thereof by medical personnel users, i.e., physicians, and yet are still effective for various endoscopic surgical procedures.

### SUMMARY OF THE INVENTION

With the foregoing in mind, embodiments of the present invention advantageously provide embodiments of a septum valve having a unique design to provide a secured septum seal around a plurality of tools that individually and separately extend through the septum valve. Embodiments of a septum valve provide an easier insertion and retraction of various laparoscopic surgical instruments as well as other surgically related items which have varying diameters. Problematical instruments do not get obstructed or caught in a multi-component valve assembly as disclosed in the prior art. Embodiments of the present invention also advantageously provide a trocar system having relatively low costs associated with the manufacturing of components of the system, e.g., valves, and thereby reduces the cost associated with the trocar system. Embodiments of the present invention additionally advantageously provide a more flexible trocar system which is effective during various endoscopic surgical procedures. The present invention further advantageously provides enhanced methods of forming a septum seal around tools and of using a trocar system during surgical procedures. Still further, because embodiments of a septum valve have a relatively flat and thin profile and because peripheries of a septum valve are fixedly connected to a valve housing, the septum valve advantageously can operate like a fixed membrane. Furthermore, because various types and diameters of tools can be used by medical personnel, embodiments of a septum valve advantageously allow one type of valve, cannula, or trocar system to be readily used for all of these various sizes and types of tools.

More particularly, a septum valve is provided which has peripheries thereof adapted to be fixedly positioned in a valve housing of a trocar system. The septum valve advantageously includes a valve body having an annular-shaped valve opening positioned in a medial portion of the valve body and adapted to individually and separately receive a plurality of different elongate tools each having a different diameter therethrough and a periphery valve section connected to and extending radially outwardly from peripheries of the valve body and having an outer perimeter thereof adapted to be fixedly connected to the valve housing. When any one of the plurality of elongate tools is positioned through the valve opening, a septum seal is maintained between peripheries of the valve body surrounding the valve opening and abuttingly contacting outer peripheries of the any one of the plurality of elongate tools extending therethrough. The valve body advantageously has first and second layers of a fabric material and a layer of elastomeric material positioned between and contacting each of the first and second layers of the fabric material. The periphery valve section advantageously has a plurality of rib members each radially extending substantially an entire distance between an outer perimeter of the valve body and the outer perimeter of the periphery valve section. The plurality of rib members are symmetrically positioned spaced-apart from each other. The periphery valve section advantageously has a greater flexibility than the valve body.

Still more particularly, a septum valve is provided for a trocar system. The septum valve advantageously includes a valve body having a valve opening adapted to individually and separately receive a plurality of different elongate tools each having a different diameter therethrough and a periphery valve section connected to and extending radially outwardly from peripheries of the valve body. When any one of the plurality of elongate tools is positioned through the valve opening, a septum seal is maintained between peripheries of the valve body surrounding the valve opening and abuttingly contacting outer peripheries of the any one of the plurality of elongate tools extending therethrough. The valve body advantageously has at least one layer of a fabric material and a layer of elastomeric material. The periphery valve section advantageously has a plurality of rib members each radially extending substantially an entire distance between an outer perimeter of the valve body and an outer perimeter of the periphery valve section.

Still more particularly, a cap assembly of a trocar system is provided which advantageously includes a substantially annular-shaped valve housing having a first opening at a proximal end and a second opening at a distal end, a first valve positioned adjacent the first opening of the valve housing and fixedly positioned entirely within the valve housing, and a second valve positioned spaced-apart from the first valve and adjacent the second opening of the valve housing. The first valve advantageously includes a valve body having an annular-shaped valve opening positioned in a medial portion of the valve body and adapted to individually and separately receive a plurality of different elongate tools each having a different diameter therethrough, and a periphery valve section connected to and extending radially outwardly from peripheries of the valve body and having an outer perimeter thereof defining the outer perimeter of the valve fixedly connected to the valve housing. When any one of the plurality of elongate tools is positioned through the valve opening, a septum seal is maintained between peripheries of the valve body surrounding the valve opening and abuttingly contacting outer peripheries of the any one of the plurality of elongate tools extending therethrough. The valve body further advantageously has first and second layers of a fabric material and a layer of elastomeric material positioned between and contacting each of the first and second layers of the fabric material. The periphery valve section advantageously has a plurality of rib members each radially extending substantially an entire distance between an outer perimeter of the valve body and the outer perimeter of the periphery valve section. The plurality of rib members are symmetrically positioned spaced-apart from each other. The periphery valve section advantageously has a greater flexibility than the valve body.

The second valve advantageously has an annular flange portion positioned within the valve housing, annular-shaped sidewalls connected to the annular flange and extending distally when positioned in the valve housing, and at least a pair of valve flaps connected to and extending inwardly from the sidewalls and flange portion. The flange portion advantageously retains portions of the second valve within the valve housing.

Still more particularly, a cap assembly of a trocar system is provided which advantageously has a valve housing having at least one opening, and at least one valve positioned adjacent the at least one opening of the valve housing. The at least one valve advantageously includes a valve body having a valve opening adapted to individually and separately receive a plurality of different elongate tools each having a different diameter therethrough, and a periphery valve section connected to and extending radially outwardly from peripheries of the valve body. When any one of the plurality of elongate tools is positioned through the valve opening, a septum seal is maintained between peripheries of the valve body surrounding the valve opening and abuttingly contacting outer peripheries of the any one of the plurality of elongate tools extending therethrough. The valve body also advantageously has at least one layer of a fabric material and a layer of elastomeric material. The periphery valve section advantageously has a plurality of rib members each radially extending substantially an entire distance between an outer perimeter of the valve body and an outer perimeter of the periphery valve section.

Further more particularly, a trocar system is provided which includes a cannula having an elongate cannula body, the cannula body having medial and distal portions thereof having a first diameter and a proximal portion thereof connected to the medial portion and having a second diameter. The second diameter is advantageously larger than the first diameter. The system also includes a valve housing which is readily detachably connected to the proximal portion of the cannula body, and at least one septum valve positioned in the valve housing and having an outer perimeter thereof fixedly connected to the valve housing. The at least one septum valve advantageously includes a valve body having an annular-shaped valve opening positioned in a medial portion of the valve body and adapted to individually and separately receive a plurality of different elongate tools each having a different diameter therethrough, and a periphery valve section connected to and extending radially outwardly from peripheries of the valve body and having an outer perimeter thereof defining the outer perimeter of the septum valve fixedly connected to the valve housing. When any one of the plurality of elongate tools is positioned through the valve opening, a septum seal is maintained between peripheries of the valve body surrounding the valve opening and abuttingly contacting outer peripheries of the any one of the plurality of elongate tools extending therethrough. The valve body advantageously has first and second layers of a fabric material and a layer of elastomeric material positioned between and contacting each of the first and second layers of the fabric material. The periphery valve section advantageously has a plurality of rib members each radially extending substantially an entire distance between an outer perimeter of the valve body and the outer perimeter of the periphery valve section. The plurality of rib members are symmetrically positioned spaced-apart from each other. The periphery valve section advantageously has a greater flexibility than the valve body.

The trocar system further includes a plurality of tools each having an elongate body for extending through the valve housing, the valve opening of the at least one septum valve, and the cannula.

A method of forming a septum valve for a trocar system is advantageously provided. The method advantageously includes providing a slab of an elastomeric material, a first layer of a fabric material overlying the elastomeric material and a second layer of a fabric material underlying the elastomeric material, cutting a disc shape in the slab, compressing the slab so that the elastomeric material extends outwardly from peripheries of the first and second layers of the fabric material, and curing the compressed slab to form a septum valve for a trocar system.

Still more particularly, a method of constructing a cap assembly for a trocar system is advantageously provided. The method advantageously includes inserting a septum valve as disclosed above into a valve housing and placing a compression ring into the valve housing adjacent the septum valve. The compression ring advantageously has an ultraviolet bonding agent associated therewith and abuttingly contacts the outer perimeter of the periphery valve section. The method also advantageously includes inserting a second valve into the valve housing adjacent and abuttingly contacting the compression ring and placing a seal ring into the valve housing adjacent the second valve. The seal ring advantageously has an ultraviolet bonding agent associated therewith and abuttingly contacts outer peripheries of the second valve. The method additionally includes the step of curing the assembly with ultraviolet light to finally construct a cap assembly for a trocar system.

Further more particularly, a method of using a trocar system is advantageously provided. The method advantageously includes providing a cap assembly which comprises a septum valve as described above and inserting a tool through the septum valve and cap assembly comprising the septum valve thereof. During the insertion process, the periphery valve section is deformed temporarily so that the valve body extends distally by contact pressure from the tool and so that a distal end of the tool is guided toward the valve opening. Afterwards, the periphery valve section is retracted to its selected biased position upon the complete insertion of the tool. The method also advantageously includes extending the tool through a cannula body matingly connected to the cap assembly at a proximal portion thereof, detaching the cap assembly from the proximal portion of the cannula body, and removing tissue or other specimen from the cannula body.

Advantageously, the various embodiments of a septum valve of the present invention provide much enhanced performance, more flexibility and better durability, and do not tear or puncture easily. These embodiments can be formed of elastomeric and fabric materials and have little mechanical problems. Also, these embodiments of a septum valve have a thin and flat profile and are easy to assemble within a valve housing. Because the septum valve is fixedly positioned within the valve housing, embodiments of a trocar system can have a septum valve which is easy for medical personnel to use. Consequently, for example, the entire cap assembly is removable from the cannula as one unit.

Because embodiments of a septum valve has a relatively flat and thin profile and because peripheries of a septum valve are fixedly connected to a valve housing, the septum valve advantageously can operate like a fixed membrane that flexes distally toward a cannula to allow tools used with the valve to be guided toward and readily inserted into the valve opening of the septum valve. Yet, because tools can be sharp or point on distal ends thereof, the fabric or reinforced layers protect the membrane operation from tears or punctures during insertion of a tool.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 is a perspective environmental view of a trocar system positioned within a layer of epidermis of a patient according to an embodiment of the present invention;

FIG. 2 is a fragmentary sectional view of a trocar system having first and second valves taken along line 2--2 of FIG. 1 according to an embodiment of the present invention;

FIG. 3 is a side elevational view of a trocar system according to an embodiment of the present invention;

FIG. 4 is a top plan view of a cap assembly of a trocar system according to an embodiment of the present invention;

FIG. 5 is an exploded view of a cap assembly of a trocar system according to an embodiment of the present invention;

FIG. 6 is a perspective view of a septum valve of a trocar system according to an embodiment of the present invention;

FIG. 7 is a top plan view of a septum valve of a trocar system according to an embodiment of the present invention;

FIG. 8 is a bottom plan view of a septum valve of a trocar system according to an embodiment of the present invention;

FIG. 9A is sectional view of a septum valve of a trocar system taken along line 9A--9A of FIG. 7 according to an embodiment of the present invention;

FIG. 9B is an enlarged fragmentary sectional view of a septum valve of a trocar system according to an embodiment of the present invention;

FIG. 10 is an environmental perspective view of a septum valve, a valve mold, and a slab illustrating the formation of a septum valve according to embodiments of the present invention;

FIG. 11 is a perspective view of an alternate embodiment of a septum valve of a trocar system according to an alternate embodiment of the present invention;

FIG. 12 is an enlarged fragmentary sectional view of an alternate embodiment of a septum valve of a trocar system taken along line 12--12 of FIG. 11 according to an alternate embodiment of the present invention;

FIG. 13 is a perspective view of an alternate embodiment of a septum valve of a trocar system according to an alternate embodiment of the present invention;

FIG. 14 is an enlarged fragmentary sectional view of an alternate embodiment of a septum valve of a trocar system taken along line 14--14 of FIG. 13 according to an alternate embodiment of the present invention;

FIG. 15A is a fragmentary perspective view of a septum valve, a valve housing and a tool illustrating the insertion of the tool into a valve opening of the septum valve according to an embodiment of the present invention;

FIG. 15B is a fragmentary perspective view of a septum valve, a valve housing and a tool illustrating the insertion of the tool into a valve opening of the septum valve according to an embodiment of the present invention; and

FIG. 15C is a fragmentary perspective view of a septum valve, a valve housing and a tool illustrating the insertion of the tool into a valve opening of the septum valve according to an embodiment of the present invention.

### DETAILED DESCRIPTION

The present invention will now be described more fully hereinafter with reference to the accompanying drawings, which illustrate embodiments of the invention. This invention may, however, be embodied in many different forms and should not be construed as limited to the illustrated embodiments set forth herein. Rather, these embodiments are provided so that this disclosure will be thorough and complete, and will fully convey the scope of the invention to those skilled in the art. Like numbers refer to like elements throughout, the prime or double prime notation, if used, indicates similar elements in alternative embodiments.

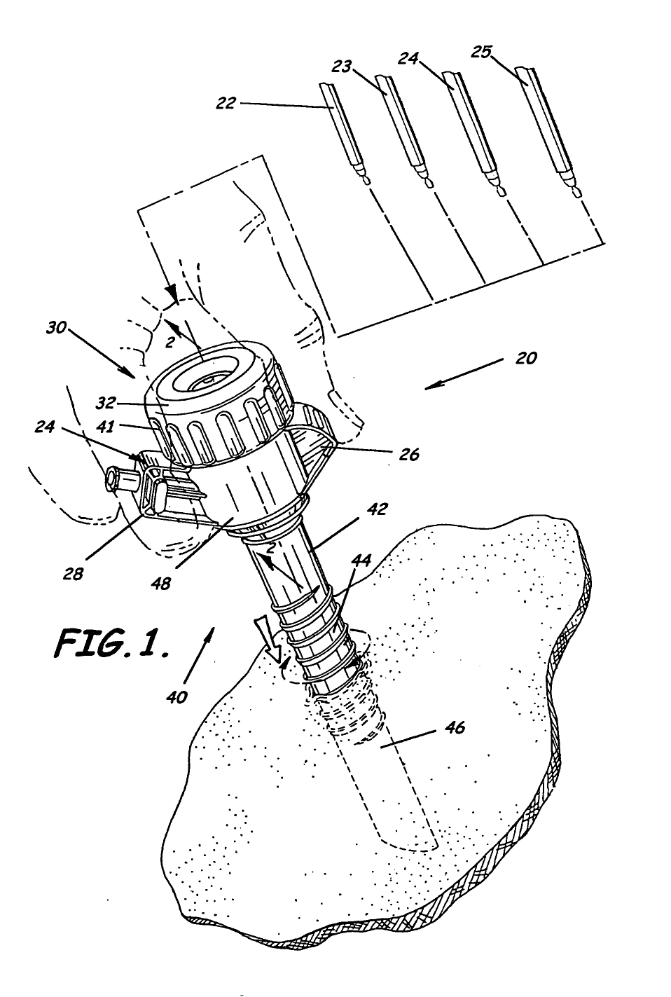

FIG. 1 illustrates a trocar system 20, which advantageously includes a cap assembly 30 having a valve housing 32. The valve housing 32 advantageously has a roughened outer surface, e.g., a plurality of dimples 41, to enhance gripping and rotating thereof by a hand of a user. The trocar system 20 also includes a cannula 40 having an elongate cannula body 42. The cannula body 42 advantageously includes distal 46 and medial 44 portions thereof having a first diameter and a proximal portion 48 thereof connected to the medial portion 44 and having a second diameter. The second diameter is advantageously larger or greater than the first diameter as illustrated. The finger gripping means 24 of the trocar system 20, for example, can be provided by a pair of finger grips 26, 28 connected to outer surfaces of the proximal portion 48 of the cannula body 42.

As illustrated in FIG. 1, the trocar system 20 further includes a plurality of tools 22, 23, 24, 25 each having an elongate body for extending through the cap assembly 30 and cannula 40. Advantageously, the plurality of tools 22, 23, 24, 25 each has a different diameter in the range of from about 4 millimeters to about 13 millimeters. The tools can be obturators or other endoscopic related tools for various endoscopic procedures.

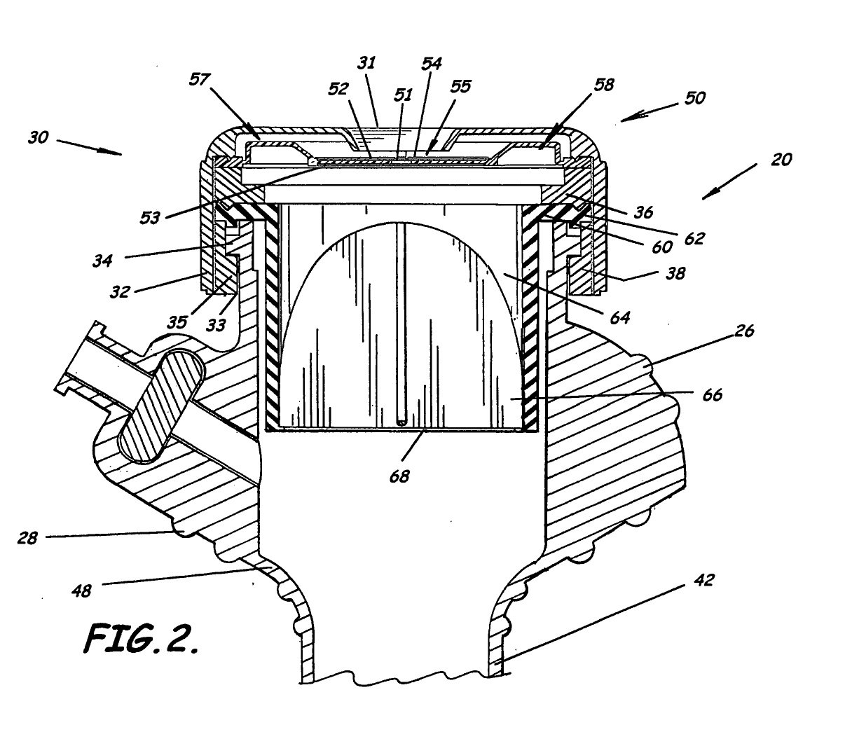

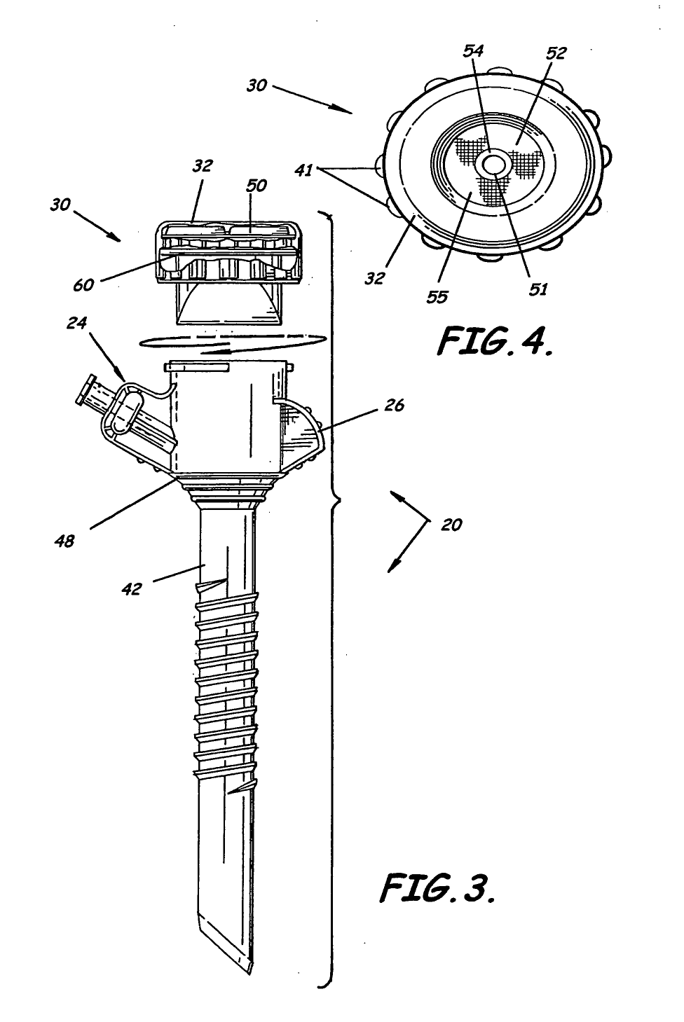

As perhaps best shown in FIGS. 1-9B, the cap assembly 30 includes a valve housing 32 which has a substantially annular shape, a first opening 31 at a proximal end, and a second opening 33 at a distal end. The cap assembly 30 also includes a septum valve 50. The septum valve 50 is positioned adjacent the first opening 31 of the valve housing 32 and has an outer perimeter thereof fixedly connected to the valve housing 32. As illustrated in FIG. 2-3, the septum valve 50 is positioned entirely within the valve housing 32.

The septum valve 50 advantageously includes a valve body 55, which has an annular-shaped valve opening 51 positioned in a medial portion of the valve body 55. The valve opening 51 is adapted to individually and separately receive a plurality of different elongate tools each having a different diameter therethrough. With such design, when any one of the plurality of elongate tools is positioned in the cap assembly 30 through the valve opening 51, a septum seal is maintained between peripheries of the valve body 55 surrounding the valve opening 51 and abuttingly contacting outer peripheries of the any one of the plurality of elongate tools extending therethrough. Additionally, the valve body 55 advantageously has first 52 and second 53 layers of a fabric material and a layer of elastomeric material 54 positioned between and contacting each of the first 52 and second 53 layers of the fabric material.

The septum valve 50 also advantageously includes a periphery valve section 57. The periphery valve section 57 is connected to and extending radially outwardly from peripheries of the valve body 55. The periphery valve section 57 has an outer perimeter thereof defining the outer perimeter of the septum valve 50, which is fixedly connected to the valve housing 32. Additionally, the periphery valve section 57 has a plurality of rib members 59 radially extending substantially an entire distance between an outer perimeter of the valve body 55 and the outer perimeter of the periphery valve section 57. Advantageously, the rib members 59 are symmetrically positioned spaced-apart from each other. Still additionally, the periphery valve section 57 further has a plurality of convolutes 58 each positioned between and connected to any two adjacent rib members. The plurality of convolutes 58 are in a selected biased position before and after each of the plurality of different elongate tools extends through the valve opening 51 individually and separately. The plurality of convolutes 58 extend toward the proximal end of the valve housing 32 when in their biased position.

As illustrated in FIG. 2-3, the cap assembly 30 also includes a second valve 60. The second valve 60 is advantageously positioned spaced-apart from the septum valve 50 and adjacent the second opening 33 of the valve housing 32. The second valve 60 advantageously has an annular flange portion 62 for enhancing the positioning of the second valve 60 within the valve housing 32, annular-shaped sidewalls 64 connected to the annular flange 62 and extending distally when positioned in the valve housing 32, and at least a pair of valve flaps 66 connected to and extending inwardly from the sidewalls 64 and flange portion 62. The sidewalls 64, for example, can extend distally of the end housing so that the flange portion 62 retains only portions of the valve 60 within the end housing 32 and yet slidably or in a spaced-apart relation have other portions which are positioned within the proximal portion 48 of the cannula body 42. The pair of valve flaps 66 have at least one slit 68 along common peripheral edges thereof through which the plurality of tools extend individually and separately. The second valve 60 also advantageously has ribs or rib members connected to, e.g., formed integrally therewith as a single piece, the sidewalls 64 as illustrated to reduce drag as will be understood by those skilled in the art. The second valve 60 is also advantageously impregnated with a lubricant such as an oil material to enhance performance of the valve.

For the septum valve 50, the periphery valve section 57 is a continuous extension of the elastomeric layer 54 of the valve body 55. The periphery valve section 57 as constructed has a greater flexibility than the valve body 55. The elastomeric material advantageously includes polyisoprene or a fiberous material being impregnated with a silicon material to enhance the strength of the valve 50 and to enhance sliding and sealing of the plurality of tools. The fabric material advantageously includes a family of high-strength and resilient synthetic polymers containing polyurethane. One example of the fabric materials that can be used for constructing the septum valve is Spandex. For Spandex, there are three possible weaves to the fabric, which essentially incorporates Nylon and Lycra in an equally balanced bi-directional weave. The combination of elastomeric and fabric materials provides an enhanced recovery memory and resiliency. As constructed, the septum valve 50 advantageously has a stretching or elastic range to readily accommodate, e.g., auto-reduction, tools or other instruments having a diameter of about 4 millimeters to about 13 millimeters as understood by those skilled in the art while still maintaining pneumoperitoneum. The valve opening 51 of the valve body 55 has a diameter less than the diameter of each of the plurality of tools that extends through the septum valve 50 so that a secured septum seal is provided around outer peripheries of each of the plurality of tools. The second valve 60 advantageously has this range as well, but individually can even have a greater range, e.g., 0 mm to 13 or 14 mm. Accordingly, with the septum valve 50 and second valve 60 in combination, the trocar system advantageously can receive different diameter instruments without the necessity of switching cannulas or valve systems.

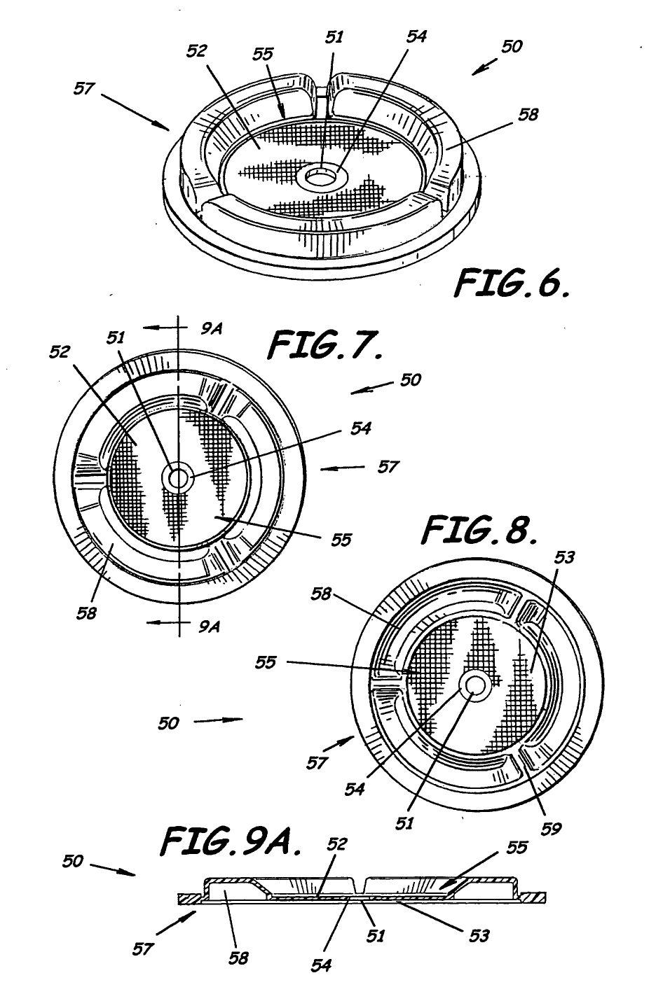

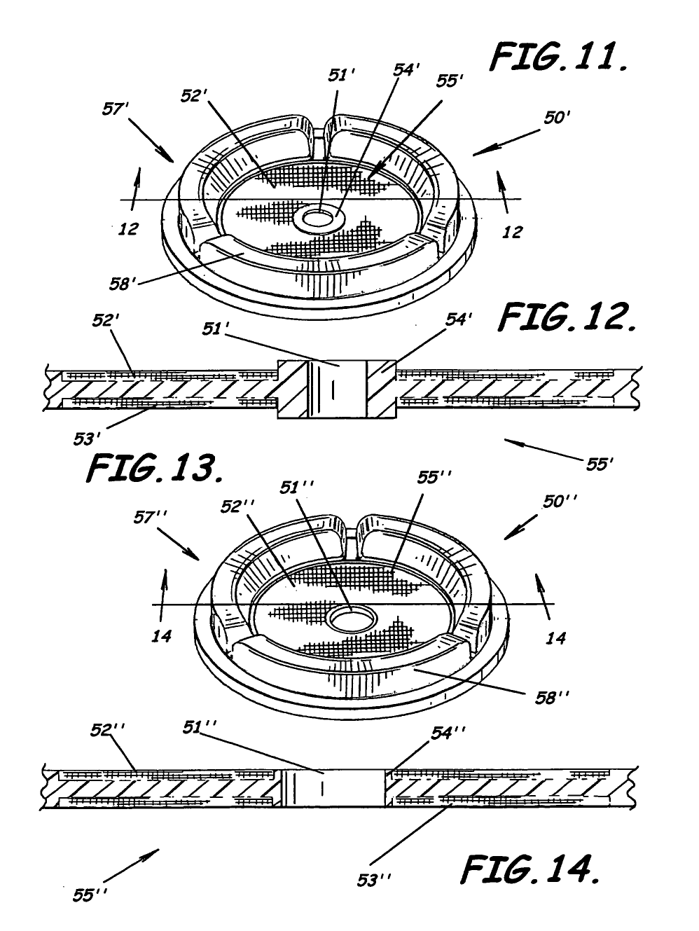

The septum valve can advantageously have various embodiments. As illustrated in FIGS. 6-9B and 11-14, wiper region, which is the medial region of the valve body surrounding the peripheries of the valve opening and is formed of the elastomeric material, can have different thickness. In one embodiment, the wiper region has a much greater or larger thickness than the adjacent fabric region as illustrated in FIGS. 6-9B. That is, the fabric layers 52, 53 have much greater or larger peripheries adjacent the valve opening 51 than the periphery of the elastomeric layer 54 surrounding the valve opening 51. In alternative embodiments, the wiper region is getting narrower and the thickness of the wiper is getting closer to the thickness of the adjacent fabric region as illustrated in FIGS. 11-14.

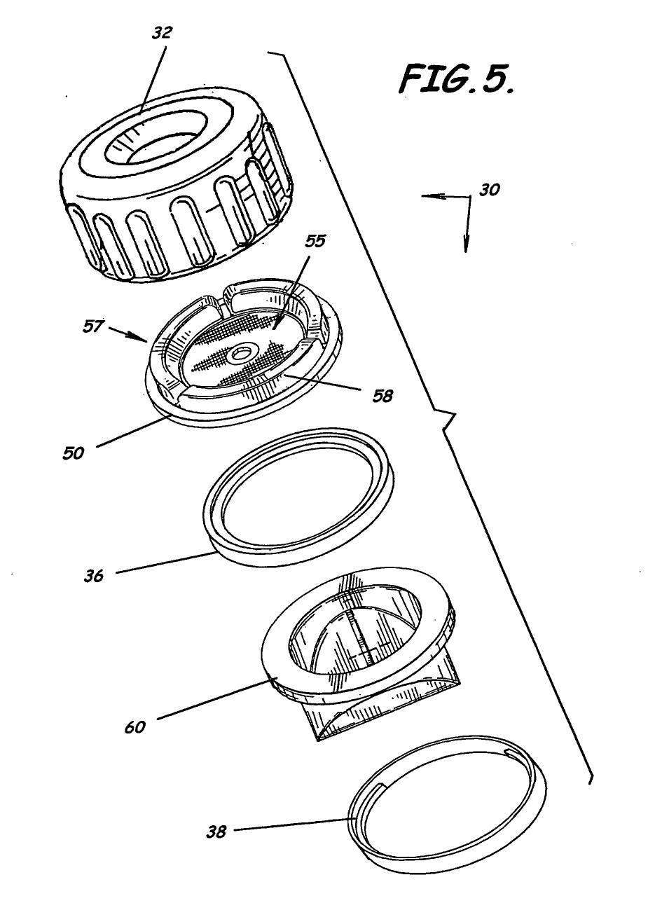

Notably, the septum valve 50 is advantageously fixedly secured to the valve housing 32. FIG. 5 illustrates a construction process of the cap assembly 30. To provide secure sealing, the septum valve 50 having the valve body 55 and periphery valve section 57 is first inserted into the valve housing 32 with the convolutes 58 extending towards the proximal end of the valve housing 32. Then a compression ring 36 coated with an ultraviolet (UV) bonding agent is placed into the valve housing 32 adjacent and abuttingly contacting the septum valve 50 in a “stacked” fashion. Following this, the second valve 60 is inserted into the valve housing 32 adjacent and abuttingly contacting the compression ring 36, and a cap seal ring 38 coated with a UV bonding agent is placed into the valve housing 32 and abuttingly contacting outer peripheries of the second valve 60. Both the compression ring 36 and cap seal ring 38 are advantageously coated with a UV bonding agent along the outer peripheries thereof abuttingly contacting the inner peripheries of the valve housing 32.

Once each of the components is in its place, the entire cap assembly is placed in a compression system, wherein each component is compressed to its desired depth into the valve housing. At that point, a UV light is exposed to the UV bonding agent to cure the materials. The curing takes place in about 8 seconds. Upon the completion of the curing, the cap assembly 30 is formed as one unit.

When constructing a trocar system, the cap assembly 30 is abuttingly connected to the cannula 40. The proximal end portion 48 of the cannula body 42 has at least one valve housing mating portion 34 associated therewith and the valve housing 32 also has at least one cannula body mating portion 35 associated therewith so that the cap assembly 30 matingly attaches to the cannula body 42 in a secured position and whereby movement of the cap assembly 30, e.g., rotational, by a hand of a user releases, e.g., unsecures or unlocks, the respective mating portions 34, 35 for ready removal of the cap assembly 30 by the user with the septum valve 50 and second valve 60 positioned therein and so that specimens, e.g., tissue, can be readily removed from the cannula body 42 without damage by the septum valve 50 and second valve 60. The extraction of large tissue samples and/or gauze packs can be accomplished without removing the cannula from the area where various endoscopic procedures take place.

The cannula body 42 is advantageously formed of a clear plastic material so that direct visualization of specimen removal and instrument passage can be advantageously provided. This, for example, allows various types of cutting, gripping, or other types of tools to be inserted through the cannula 40 for various endoscopic procedures.

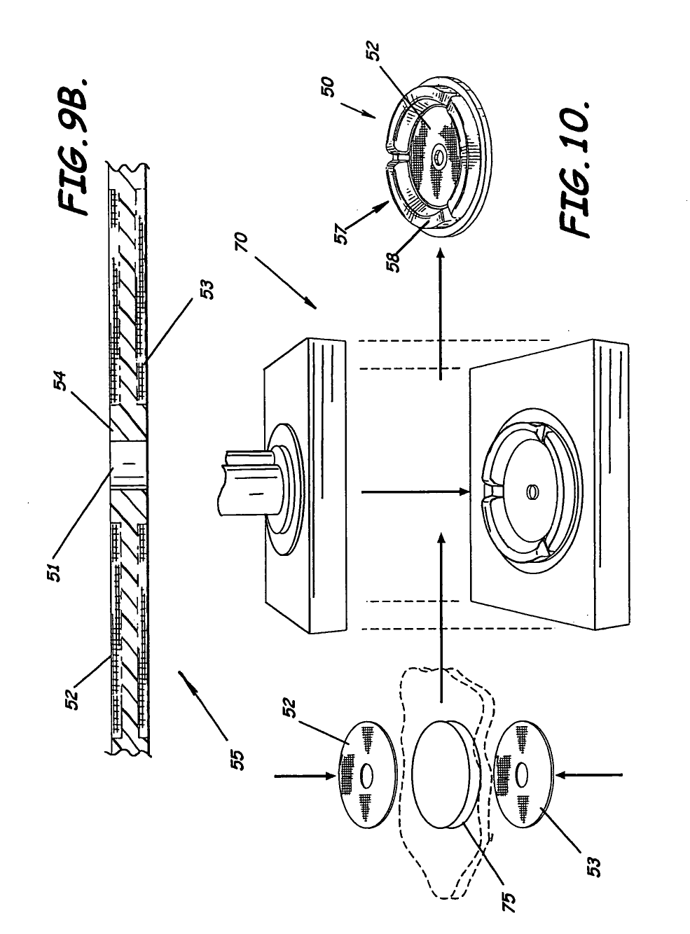

As illustrated in FIG. 10, the present invention also advantageously includes a process 70 of forming a septum valve for a trocar system. A slab of an elastomeric material 75 is advantageously provided with a first layer 52 of a fabric material overlying the elastomeric material 75 and a second layer 53 of a fabric material underlying the elastomeric material 75. Each of the first 52 and second 53 layers of the fabric material is precut with an opening in the medial portion thereof. A disc shape is cut in the medial portion of the slab thereby forming a valve body 55. The slab is then placed in a tool and compressed so that the elastomeric material 75 extends outwardly from peripheries of the first 52 and second 53 layers of the fabric material thereby forming a periphery valve section 57. The compressed slab is then cured with heat and pressure to form a septum valve. Before the compression of the slab, a piece of raw elastomeric material, e.g., polyisoprene, can be placed on top of the tool. When activated, the compression forces the raw polyisoprene into the tool to form part of the convolutes 58.

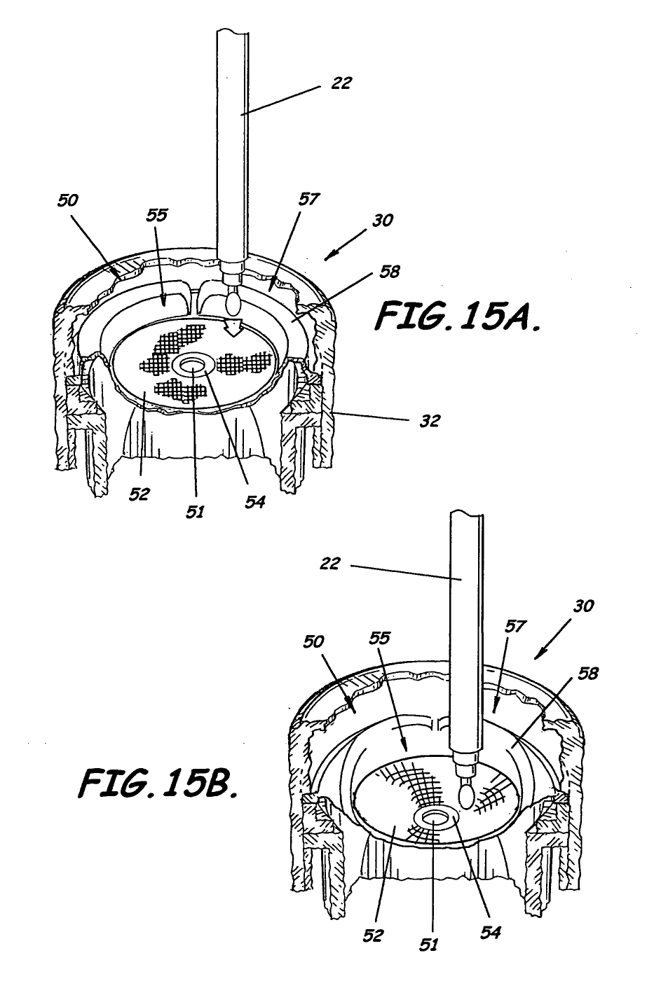

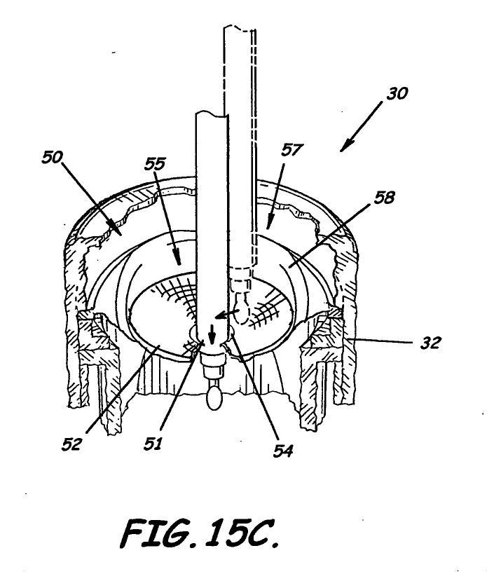

As illustrated in FIGS. 1-15C and as described above, the present invention also includes embodiments of a method of using a trocar system 20 including the steps of providing a cap assembly 30, which includes a septum valve 50, 50′, 50″, and inserting a tool 22, 23, 24, 25 through the septum valve 50, 50′, 50″ and cap assembly 30. During the insertion, the convolutes 58, 58′, 58″ of the periphery valve section 57, 5′, 57″ flex inwardly around the medial portion of the valve body 55, 55′, 55″ and consequently, the periphery valve section 57, 57′, 57″ is deformed temporarily so that the valve body 55, 55′, 55″ extends distally by contact pressure from the tool 22, 23, 24, 25 and so that a distal end of the tool 22, 23, 24, 25 is guided toward the valve opening 51, 51′, 51″ (see FIGS. 15A-15C). The unique symmetric rib structure of the periphery valve section 57, 57′, 57″ reinforces the movement of the convolutes 58, 58′, 58″ and the recovery of the convolutes 58, 58′, 58″. Because the septum valve 50, 50′, 50″ is constructed in a thin and relatively flat profile, the septum valve 50, 50′, 50″ functions like a thin elastic membrane. The membrane flexes inwardly and outwardly around the medial portion of the valve body 55, 55′, 55″ with the outer peripheries fixedly secured within the valve housing 32 and does not float or rotate in the valve housing 32. Upon the complete insertion of the tool 22, 23, 24, 25, the convolutes 58, 58′, 58″ flex outwardly around the medial portion of the valve body 55, 55′, 55″ and consequently, the periphery valve section 57, 57′, 57″ is retracted to its selected biased position. This method also includes extending the tool 22, 23, 24, 25 through a cannula body 42 matingly connected to the cap assembly 30 at a proximal portion 48 thereof, detaching the cap assembly 30 from the cannula body 42 and removing tissue or other specimen as understood by those skilled in the art from the cannula body 42.

Embodiments of the septum valve 50, 50′, 50″ includes a valve body 55, 55′, 55″ having an annular-shaped valve opening 51, 51′, 51″ positioned in a medial portion of the valve body 55, 55′, 55″ and adapted to receive a plurality of tools 22, 23, 24, 25 individually and separately therethrough. The valve body 55, 55′, 55″ advantageously has first 52, 52′, 52″ and second 53, 53′, 53″ layers of a fabric material and a layer of elastomeric material 54, 54′, 54″ positioned between and contacting each of the first and second layers of the fabric material. The septum valve 50, 50′, 50″ also includes a periphery valve section 57, 57′, 57″ connected to and extending radially outwardly from peripheries of the valve body 55, 55′, 55″ and having an outer perimeter thereof. The periphery valve section 57, 57′, 57″ advantageously has a plurality of rib members 59 each radially extending substantially an entire distance between an outer perimeter of the valve body 55, 55′, 55″ and the outer perimeter of the periphery valve section 57, 57′, 57″ and symmetrically positioned spaced-apart from each other (see FIGS. 2, 6-9B and 11-14). The periphery valve section 57, 57′, 57″ advantageously has a greater flexibility than the valve body 55, 55′, 55″.

Because embodiments of a septum valve 50, 50′, 50″, according to the present invention, have a relatively flat and thin profile and because peripheries of a septum valve 50, 50′, 50″ are fixedly connected to a valve housing 32, the septum valve 50, 50′, 50″ advantageously can operate like a fixed membrane that flexes distally toward the cannula to allow tools 22, 23, 24, 25 used with the valve 50, 50′, 50″ to be guided toward and readily inserted into the valve opening 51, 51′, 51″. Yet, because tools 22, 23, 24, 25 can be sharp or point on distal ends thereof, the fabric or reinforced layers protect the membrane operation from tears or punctures during insertion of a tool 22, 23, 24, 25. Further, because various types and diameters of tools can be used by medical personnel, embodiments of a septum valve advantageously allow one type of valve, cannula, or trocar system to be readily used for all of these various sizes and types of tools.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

1. A septum valve having peripheries thereof adapted to be fixedly positioned in a valve housing of a trocar system comprising:
a valve body having an annular-shaped valve opening positioned in a medial portion of the valve body and adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having first and second layers of a fabric material and a layer of elastomeric material positioned between and contacting each of the first and second layers of the fabric material; and
a periphery valve section connected to and extending radially outwardly from peripheries of the valve body and having an outer perimeter thereof adapted to be fixedly connected to the valve housing, the periphery valve section having a plurality of rib members each radially extending substantially an entire distance between an outer perimeter of the valve body and the outer perimeter of the periphery valve section and symmetrically positioned spaced-apart from each other, the periphery valve section having a greater flexibility than the valve body.

2. The septum valve of claim 1, wherein each of the plurality of elongate tools has a diameter in the range of about 4 millimeters to 13 millimeters, and wherein the annular-shaped valve opening has a diameter less than about 4 millimeters.

3. The septum valve of claim 1, wherein the periphery valve section further has a plurality of convolutes each positioned between and connected to any two adjacent rib members, the plurality of convolutes each extending toward a proximal end of the valve housing and being in a selected biased position before and after individually and separately receiving a plurality of different elongate tools through the valve opening.

4. The septum valve of claim 1, wherein the periphery valve section is formed of the same elastomeric material as the elastomeric layer of the valve body.

5. The septum valve of claim 1, wherein the layer of elastomeric material is intermeshed with the first and second layers of the fabric material of the valve body, the fabric material including at least one of a family of resilient synthetic polymers containing polyurethane, the elastomeric material including polyisoprene and silicone.

6. A septum valve comprising:
a valve body having a valve opening adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having at least one layer of a fabric material and a layer of elastomeric material; and
a periphery valve section connected to and extending radially outwardly from peripheries of the valve body, the periphery valve section having a plurality of rib members each radially extending substantially an entire distance between an outer perimeter of the valve body and an outer perimeter of the periphery valve section.

7. The septum valve of claim 6, wherein the valve opening has a substantially annular shape and is positioned in a medial portion of the valve body, and wherein the valve opening has a diameter less than the diameter of each of the plurality of tools.

8. The septum valve of claim 7, wherein each of the plurality of elongate tools has a diameter in the range of about 4 millimeters to 13 millimeters, and wherein the annular-shaped valve opening has a diameter less than about 4 millimeters.

9. The septum valve of claim 6, wherein the plurality of rib members are symmetrically positioned spaced-apart from each other.

10. The septum valve of claim 9, wherein the periphery valve section further has a plurality of convolutes each positioned between and connected to any two adjacent rib members, the plurality of convolutes being in a selected biased position before and after individually and separately receiving a plurality of different elongate tools through the valve opening.

11. The septum valve of claim 6, wherein the periphery valve section is formed of the same elastomeric material as the elastomeric layer of the valve body.

12. The septum valve of claim 6, wherein the layer of elastomeric material is intermeshed with the at least one layer of the fabric material in the valve body, the fabric material including at least one of a family of resilient synthetic polymers containing polyurethane, the elastomeric material including polyisoprene and silicone.

13. The septum valve of claim 12, wherein the layer of elastomeric material is at least as thick as the at least one layer of the fabric material around the valve opening of the valve body.

14. A cap assembly of a trocar system comprising:
a valve housing having a substantially annular shape, a first opening at a proximal end, and a second opening at a distal end;
a first valve positioned adjacent the first opening of the valve housing and fixedly positioned entirely within the valve housing, the first valve having an outer perimeter thereof fixedly connected to the valve housing, the first valve including a valve body having an annular-shaped valve opening positioned in a medial portion of the valve body and adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having first and second layers of a fabric material and a layer of elastomeric material positioned between and contacting each of the first and second layers of the fabric material, and a periphery valve section connected to and extending radially outwardly from peripheries of the valve body and having an outer perimeter thereof defining the outer perimeter of the valve fixedly connected to the valve housing, the periphery valve section having a plurality of rib members each radially extending substantially an entire distance between an outer perimeter of the valve body and the outer perimeter of the periphery valve section and symmetrically positioned spaced-apart from each other, the periphery valve section having a greater flexibility than the valve body; and
a second valve positioned spaced-apart from the first valve and adjacent the second opening of the valve housing, the second valve having an annular flange portion positioned within the valve housing, annular-shaped sidewalls connected to the annular flange and extending distally when positioned in the valve housing, and at least a pair of valve flaps connected to and extending inwardly from the sidewalls and flange portion, the flange portion retaining portions of the second valve within the valve housing.

15. The cap assembly of claim 14, wherein each of the plurality of elongate tools has a diameter in the range of about 4 millimeters to 13 millimeters, and wherein the annular-shaped valve opening has a diameter less than about 4 millimeters.

16. The cap assembly of claim 14, wherein the periphery valve section of the first valve further has a plurality of convolutes each positioned between and connected to any two adjacent rib members, the plurality of convolutes each extending toward the proximal end of the valve housing and being in a selected biased position before and after individually and separately receiving a plurality of different elongate tools through the valve opening.

17. The cap assembly of claim 14, wherein the periphery valve section is formed of the same elastomeric material as the elastomeric layer of the valve body.

18. The cap assembly of claim 14, wherein the layer of elastomeric material is intermeshed with the first and second layers of the fabric material in the valve body, the fabric material including at least one of a family of resilient synthetic polymers containing polyurethane, the elastomeric material including polyisoprene and silicone.

19. A cap assembly of a trocar system comprising:
a valve housing having at least one opening; and
at least one valve positioned adjacent the at least one opening of the valve housing, the at least one valve including a valve body having a valve opening adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having at least one layer of a fabric material and a layer of elastomeric material, and a periphery valve section connected to and extending radially outwardly from peripheries of the valve body, the periphery valve section having a plurality of rib members each radially extending substantially an entire distance between an outer perimeter of the valve body and an outer perimeter of the periphery valve section.

20. The cap assembly of claim 19, wherein the valve opening of the at least one valve has a substantially annular shape and is positioned in a medial portion of the valve body, and wherein the valve opening has a diameter less than the diameter of each of the plurality of tools.

21. The cap assembly of claim 20, wherein each of the plurality of elongate tools has a diameter in the range of about 4 millimeters to 13 millimeters, and wherein the annular-shaped valve opening has a diameter less than about 4 millimeters.

22. The cap assembly of claim 19, wherein the plurality of rib members of the periphery valve section of the at least one valve are symmetrically positioned spaced-apart from each other.

23. The cap assembly of claim 19, wherein the periphery valve section of the at least one valve further has a plurality of convolutes each positioned between and connected to any two adjacent rib members, the plurality of convolutes each extending toward the proximal end of the valve housing and being in a selected biased position before and after individually and separately receiving a plurality of different elongate tools through the valve opening.

24. The cap assembly of claim 19, wherein the periphery valve section is formed of the same elastomeric material as the elastomeric layer of the valve body.

25. The cap assembly of claim 19, wherein the layer of elastomeric material is intermeshed with the at least one layer of the fabric material in the valve body, the fabric material including at least one of a family of resilient synthetic polymers containing polyurethane, the elastomeric material including polyisoprene and silicone.

26. A trocar system comprising:
a cannula having an elongate cannula body, the cannula body including medial and distal at least portions thereof having a first diameter and a proximal portion thereof connected to the medial portion and having a second diameter, the second diameter being greater than the first diameter;
a valve housing being readily detachably connected to the proximal portion of the cannula body;
at least one valve positioned in the valve housing and having an outer perimeter thereof fixedly connected to the valve housing, the at least one valve including a valve body having an annular-shaped valve opening positioned in a medial portion of the valve body and adapted to individually and separately receive a plurality of different elongate tools each having a different diameter therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having first and second layers of a fabric material and a layer of elastomeric material positioned between and contacting each of the first and second layers of the fabric material, and a periphery valve section connected to and extending radially outwardly from peripheries of the valve body and having an outer perimeter thereof defining the outer perimeter of the septum valve fixedly connected to the valve housing, the periphery valve section having a plurality of rib members each radially extending substantially an entire distance between an outer perimeter of the valve body and the outer perimeter of the periphery valve section and symmetrically positioned spaced-apart from each other, the periphery valve section having a greater flexibility than the valve body; and
a plurality of tools each having an elongate body for extending through the valve housing, the valve opening of the at least one valve, and the cannula.

27. The trocar system of claim 26, wherein the valve housing has a substantially annular shape and first and second openings, the first opening positioned at a proximal end and adjacent the at least one septum valve, the second opening positioned at a distal end and having at least one cannula body mating portion associated therewith and wherein the proximal portion of the cannula body has at least one valve housing mating portion associated therewith so that the cannula body can matingly attach to the valve housing in a secured position.

28. The trocar system of claim 26, further comprising:
a second valve positioned adjacent the second opening of the valve housing, the second valve having an annular flange portion positioned within the valve housing, annular-shaped sidewalls connected to the annular flange and extending distally when positioned in the valve housing, and at least a pair of valve flaps connected to and extending inwardly from the sidewalls and flange portion, the flange portion retaining at least portions of the second valve within the valve housing.

29. The trocar system of claim 26, wherein the periphery valve section further has a plurality of convolutes each positioned between and connected to any two adjacent rib members, the plurality of convolutes each extending toward a proximal end of the valve housing and being in a selected biased position before and after individually and separately receiving a plurality of different elongate tools through the valve opening.

30. The trocar system of claim 26, wherein the periphery valve section is formed of the same elastomeric material as the elastomeric layer of the valve body.

31. The trocar system of claim 26, wherein the layer of elastomeric material is intermeshed with the first and second layers of the fabric material in the valve body, the fabric material including at least one of a family of resilient synthetic polymers containing polyurethane, the elastomeric material including polyisoprene and silicone.

32. A method of forming a septum valve for a trocar system, comprising the steps of:
providing a slab of an elastomeric material, a first layer of a fabric material overlying the elastomeric material and a second layer of a fabric material underlying the elastomeric material;
cutting a disc shape in the slab;
compressing the slab so that the elastomeric material extends outwardly from peripheries of the first and second layers of the fabric material; and
curing the compressed slab to form a septum valve for a trocar system.

33. A method of constructing a cap assembly for a trocar system, comprising the steps of:
inserting a septum valve into a valve housing, the septum valve including a valve body having an annular-shaped valve opening positioned in a medial portion of the valve body and adapted to receive individually and separately a plurality of tools therethrough so that when any one of the plurality of elongate tools is positioned through the valve opening a septum seal is maintained between peripheries of the valve body surrounding the valve opening and abuttingly contacting outer peripheries of the any one of the plurality of elongate tools extending therethrough, the valve body having first and second layers of a fabric material and a layer of elastomeric material positioned between and contacting each of the first and second layers of the fabric material, and a periphery valve section connected to and extending radially outwardly from peripheries of the valve body and having an outer perimeter thereof adapted to be fixedly connected to the valve housing, the periphery valve section having a plurality of rib members each radially extending substantially an entire distance between an outer perimeter of the valve body and the outer perimeter of the periphery valve section and symmetrically positioned spaced-apart from each other, the periphery valve section having a greater flexibility than the valve body;
placing a compression ring into the valve housing adjacent the septum valve, the compression ring having an ultraviolet bonding agent associated therewith and abuttingly contacting the outer perimeter of the periphery valve section;
inserting a second valve into the valve housing adjacent and abuttingly contacting the compression ring;
placing a seal ring into the valve housing adjacent the second valve, the seal ring having an ultraviolet bonding agent associated therewith and abuttingly contacting outer peripheries of the second valve; and
curing the assembly with ultraviolet light to construct a cap assembly for a trocar system.

34. A method of using a trocar system, comprising the steps of:
providing a cap assembly in a trocar system, the cap assembly comprising a septum valve including a valve body having an annular-shaped valve opening positioned in a medial portion of the valve body and adapted to receive individually and separately a plurality of tools therethrough so that when any one of the plurality of tools is positioned through the valve opening a septum seal is maintained between peripheries of the valve body surrounding the valve opening and abuttingly contacting outer peripheries of the any one of the plurality of tools extending therethrough, the valve body having first and second layers of a fabric material and a layer of elastomeric material positioned between and contacting each of the first and second layers of the fabric material, and a periphery valve section connected to and extending radially outwardly from peripheries of the valve body and having an outer perimeter thereof adapted to be fixedly connected to the valve housing, the periphery valve section having a plurality of rib members each radially extending substantially an entire distance between an outer perimeter of the valve body and the outer perimeter of the periphery valve section and symmetrically positioned spaced-apart from each other, the periphery valve section having a greater flexibility than the valve body;
inserting a tool through the septum valve and cap assembly comprising the septum valve thereof, during which the periphery valve section is deformed temporarily so that the valve body extends distally by contact pressure from the tool and so that a distal end of the tool is guided toward the valve opening and then the periphery valve section is retracted to its selected biased position upon the complete insertion of the tool;
extending the tool through a cannula body matingly connected to the cap assembly at a proximal portion thereof;
detaching the cap assembly from the proximal portion of the cannula body; and
removing tissue or other specimen from the cannula body.
